(19) United States
(12) Patent Application Publication
(10) Pub. No.: US 2016/0128605 A1
(43) Pub. Date: May 12, 2016

(54) Apparatus for Calculating a Parameter of a Skin Surface Area

(71) Applicant: SEB S.A., Ecully (FR)

(72) Inventors: Marie-Valerie Moreno, Chindrieux (FR); Franck Mandica, Francheville (FR); Monique Paget, Optevoz (FR); Michel Sarrazin, Massingy (FR)

(21) Appl. No.: 14/893,728

(22) PCT Filed: May 22, 2014
(86) PCT No.: PCT/FR2014/051203

§ 371 Date: Nov. 24, 2015

(30) Foreign Application Priority Data
May 27, 2013 (FR) 1354773

(51) Int. Cl. A61B 5/053; A61H 15/00; A61H 9/00; A61B 5/00

(52) U.S. Cl. CPC A61B 5/0537; A61B 5/443; A61B 5/4836; A61B 5/0531; A61H 15/0085; A61H 9/0057; A61H 2015/0014; A61H 2207/00; A61H 2230/655; A61B 2560/0214

(57) ABSTRACT

Provided is a mobile autonomous apparatus (1) for measuring and analysing at least one parameter representing the fat of a skin surface area comprising at least one probe (10) comprising at least two pairs of electrodes intended to come into contact with the skin surface area defining a frrst pair of injecting electrodes (11, 12), and a second pair ofreceiving electrodes (13, 14) for measuring the response to the injection, an energy source (5) for producing, at the frrst pair of injecting electrodes (11, 12), a variable electrical signal with a current of intensity i included in the range [60 μA to 4000 μA] and a frequency included in the range [1 k Hz to 1000 kHz], a data processing and control unit (CPU) for determining the elements relative to the bio-impedance (Z) of said area and said at least one parameter representing the cellulite of the skin surface area.

This invention pertains to the technical field of calculating a physico-chemical parameter of the skin surface of a living being from a measurement based on the principle of impedancemetry, and particularly a parameter pertaining to the presence of fat, such as cellulite, for example.

### PRIOR ART

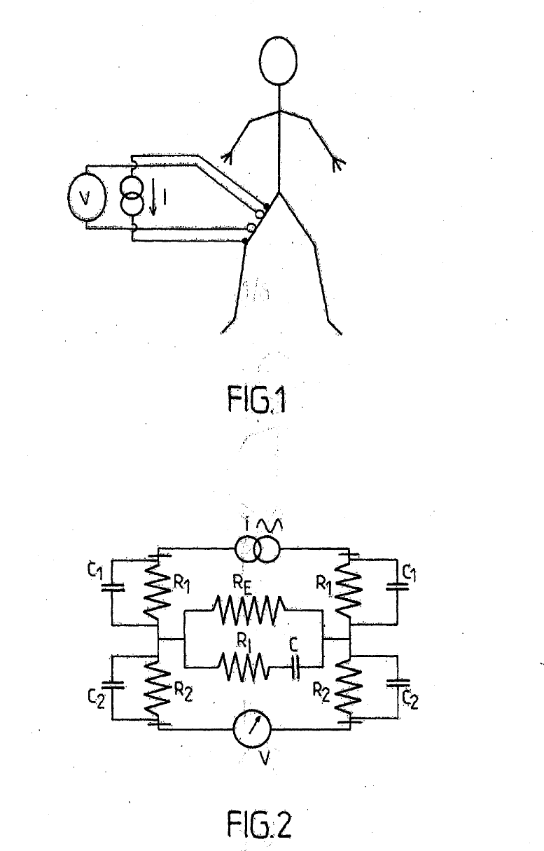

Impedancemetry is based on a measurement of the electrical impedance of biological tissue: the tissue prevents a low-intensity current from passing through. Known devices are based on a principle consisting of applying a low alternating current (i), on the order of microamperes, with a low frequency, through a pair of electrodes applied to an individual's body in a non-invasive manner, for example two or four, as illustrated in FIG. 1. The difference in potential (V) between said electrodes makes it possible to calculate the impedance (7) of the body part, which is similar to a suspension of cells in an electrolytic solution. Determining the impedance (Z) of a body part is done based on an electrical model of the body part, taking into account the frequency (f c) of the electric current used, and the resistance preventing the electric current from passing through the water and the intra- and extra-cellular electrolytes. The resistance values of said electrolytes are represented by RI and RE, respectively, in a model such as the Fricke model. Calculating bio-electrical impedance also takes into account the reactance (Xfc) of the membrane of the body part, the effect of which is to cause a phase shift (φ) between the current applied and the voltage measured. In reference to FIGS. 2, R1, R2, C1 and C2 represent the resistance values and the contact capacities of the electrodes, respectively, and the equations relating these factors are as follows:

Z²=R²+X²

X=|Z|×sin φ

R=|Z|×cos φ

Depending on the tissue in question and the measurement sought, the choice of intensity and frequency parameters is important. Depending on the electrical currents, the order of magnitude in microamperes applied and their frequency, the measurement can provide extra- or intracellular indications, as the current does or does not pass through the cells of the organism. The measurement is thus taken at a depth of a few millimeters to one centimeter, without interference from the deeper layers.

Moreover, many physiological and metabolic parameters of the cutaneous surface or skin are already known, including parameters of cellulite or hypodermal fat or particularly epidermal fat, which is characterized by the presence of fat and even water in the skin, and primarily the thigh and buttocks skin of a human being. Cellulite is the first layer of tat in terms of depth (it is approximately 1 cm deep) and forms the dimpling that visually gives the skin an “orange peel” appearance, Cellulite located in the cutaneous layers is not the fat associated with excess weight located between the skin and the muscle. Cellulite is known as a female function of the hypodermis, which causes a swelling of the adipocytes, and then in a more advanced stage, a mechanical deformity that is visible on the surface of the skin. Cellulite causes fatty protrusions at the hypodermal attachment, giving the skin this unsightly relief. Cellulite is often accompanied by water retention in the tissue. A need arose for a precise, simple measurement of cutaneous surface parameters such as cellulite in order to be able to assess cellulite and even monitor its evolution over time in an individual. The measurement is taken in particular using a bio-impedance measurement, for example.

Electrical apparatuses for treating cellulite are already known to exist, such as massage rollers, or vacuum massagers, and even vacuum massage rollers known as palpate-and-roll apparatuses. A palpate-and-roll apparatus is a massage device comprising an adjacent low-pressure chamber to create a suction of the skin, for example surrounded on each side by a massage roller that can be spun by a motor and/or slowed. The operation and results of this type of device often depend on the person being treated and may cause small bruises if the massage force is too great.

Such apparatuses already exist with a means of adjusting the force of the massage, for example the suction level that the user will program himself with one or more parameters before or during the session.

There already exists, for example in W02007051896, a “palpate-and-roll” massage apparatus comprising a base connected to a skin massaging head comprising a low-pressure chamber to create a suction of the skin, surrounded on each side by a massage roller. This apparatus comprises a sensor to measure one or more parameters of the skin and a means of automatically adjusting the suction level to a desired value based on the measurements. This involves various sensors, for example, to measure the composition of the skin, such as the fluid content, and advantageously, the fat content. Two sensors may be used to measure the fluid and fat content. The sensors can be integrated in or separate from the massage apparatus. Adjustments are based on several parameters of the skin, including a measurement of impedance, capacitance, resistance, reactance and inductance, or even the strength, flexibility, elasticity and/or composition of the tissue. An impedance spectroscopy is then performed. In any case, it is the suction level that is then adjusted and other power sources.

A need arose to better control a skin treatment apparatus, depending on the results to be achieved and/or depending on the user, or to offer adjustments and series of skin treatment sessions, such as for cellulite.

The purpose of this invention is to provide an autonomous apparatus capable of taking a “periodic” measurement of a parameter of the skin surface, such as fat, or more precisely subcutaneous abdominal or thigh fat, or cellulite as another example. This measurement apparatus pertains to the use of measurements that are periodic, or repeated at intervals. These measurements are used to determine the results of the parameter, or even, in a second aspect of the invention, to suggest a particular skin treatment or to automatically control a skin treatment apparatus, such as a cellulite treatment apparatus.

### PRINCIPLE OF THE INVENTION

A first facet of the invention pertains to an autonomous, mobile apparatus for measuring and analyzing at least one parameter representing the fat, such as hypodermal fat or cellulite, of a skin surface, while a second facet of the invention pertains to a skin treatment device including, in a built-in manner, said autonomous mobile measurement and analysis apparatus. The representation of cellulite may be done by level or stage of cellulite.

Thus the first facet of the invention pertains to an autonomous, mobile apparatus for measuring and analyzing at least one parameter representing the fat of a skin surface area, comprising at least one probe with at least two pairs of electrodes designed to come into contact with the skin surface area defining a first pair of injecting electrodes, and a second pair of receiving electrodes for measuring the response to the injection, a power source for producing in the first pair of injecting electrodes a variable electrical signal of current intensity (i) within the range [60 μA; 4000 μA] and a frequency within the range [1 Hz; 1000 kHz], for example in the sub-range [1 kHz; 1000 kHz]; a data control processing unit (CPU) for determining information related to the bio-impedance of said area and said at least one parameter representing the fat of the skin surface area. The fat may be cellulite, which is hypodermal fat or subcutaneous fat.

The intensity value range is [60 μA; 4000 μA] and more specifically will be [77 μA; 3800 μA], and even more precisely [600 μA; 800 μA]. One intensity that works particularly well is 800 μA. The frequency value range is [1 Hz; 1000 kHz], and specifically may be [1 kHz; 1000 kHz] and even [5 kHz; 350 kHz]. One frequency that works particularly well is 50 kHz. The variable alternating current may be a current with a sinusoidal signal as a function of time or a current with a rectangular signal, such as square, for example, as function of time. Measurements are taken at several frequencies. When it pertains to a current with a square signal, the current described in the applicant's patent FR2775581 may be used.

For example, from an alternating current with a square signal, one assumes that it is admissible for the characteristic resistance (Rc) to be equivalent to the resistance at 50 kHz, which is the usual resistance of subcutaneous fat equations. Now, the characteristic resistance (Rc) corresponds to the direction change resistance of the impedance circle shown in FIG. 7. Therefore, the real-part projection (on the x-axis) of this Rc corresponding to R 50 kHz is simply the average of the resistance values extrapolated to a null and infinite frequency produced by the square signal, or in other words (Ret+Rinft)/2. This is also true for a sinusoidal signal, where Ret and Rinft are the resistance values extrapolated according to the Cole-Cole model, in particular.

The following equation is thus proposed to quantify the subcutaneous fat determined by the data control processing unit (CPU), based on an alternating current with a rectangular signal, such as square, for example, or a sinusoidal signal, as being equal or proportional to:

A*((Ret+Rinft)/2)−B

Where Ret is the resistance extrapolated to a null frequency,
Rinft is the resistance extrapolated to an infinite frequency,
Where A and B are constants
With A being within [0.5; 1.5], and preferably A=0.9; with B being within [5; 15], and preferably B−10.

For cellulite or hypodermal fat, it appears that a dry-pressure local measurement with a reusable interface makes it possible to approach the thickness of the cellulite, and thus the following equation is proposed, in which the thickness of the cellulite, determined by the data control processing unit (CPU) is equal or proportional to:

Ln (Rinft/C)/D

Where Rinft is the resistance extrapolated to an infinite frequency, and where C and D are constants, In=Napierian logarithm; with C being within [15; 35], and preferably C=25; with D being within [0,01; 0.03], and preferably D=0.02. The result may be reported in a qualitative unit of the color gradient type by stage of celluiite thickness. The result could be reported in a quantitative unit, using the example here, in millimeters.

It has been observed, in a surprising manner, that cellulite is in fact related to the resistance value extrapolated to an infinite frequency (Rinft) of the real part of Z under a current within a range of 77 to 800 microamperes. This is based on the idea that cellulite is a tissue comprised of adipocytes and water, represented by Rinft.

These equations have been established through extrapolation and through testing and validation on several individuals.

The volume or the thickness of a dimple can therefore be estimated, since one has the thickness [and] one has the linear quantity of dimples.

Having the surface area measurement of the sensor, one should indeed be able to ascertain the number of dimples per cm²,

Electrode contact is non-invasive. The term “autonomous” or “independent” refers to a device that, alone, is capable of measuring and calculating the skin parameter, without the assistance of other apparatuses linked by a hard-wired or wireless connection. The autonomous apparatus has electrodes built in, and therefore does not require the attachment of electrodes to the skin. The measured skin may be that of the lower part of the body, such as the thighs, buttocks or stomach, for example, or even the upper part of the body, such as the face. The measurement is local.

In one particular aspect of the invention, one of the parameters in question, pertaining to the fat in the skin is cellulite, otherwise referred to as hypodermal fat, which represents the accumulation of fat beneath the skin. Its scientific name is EFP, for “Edematous Fibrosclerotic Panniculopathy,” which corresponds to different stages of progression of cellulite. The increase in the size of these adipocytes or the excessive infiltration of water in the tissue are the causes of an increased thickness of the hypodermis (located beneath the epidermis), illustrated by a change in the adipose system more commonly referred to as cellulite.

Today, cellulite can be characterized in several ways, as there is no parameter that is recognized as the standard. For example, one can characterize the progression of cellulite based on its volume or thickness, using a predetermined dimpling score, using its visual appearance of roughness, the hypodermal attachment size, the fat content percentage image, or a combination of said parameters, among others.

In one particular aspect of the invention, the data control processing unit (CPU) makes it possible to determine the characterization of the parameter representing the cellulite, which includes a natural integer on a scale between 0 and n, representing the stage of progression of the cellulite. The scale will be established in increasing order according to the stage of progression of the cellulite between 0 and n, using 10 as an example. One can call a stage on the scale a “dimpling score.” It is calculated based on bio-impedance measurements of the layer in question.

In one particular aspect of the invention, the data control processing unit (CPU) makes it possible to determine the characterization of the parameter representing the cellulite, which includes the length of the hypodermal attachment for a unit length of the skin area, or the hypodermal attachment size for a unit surface area of the skin area. The hypodermal attachment or HA is the attachment between the dermis and the epidermis of the skin area, and in this case it is its relief, representing cellulite, that will be characterized based on bio-impedance measurements of the layer in question.

In one particular aspect of the invention, the data control processing unit (CPU) makes it possible to determine the characterization of the parameter representing the cellulite, which is the surface roughness of the skin area. Here it is a matter of providing a representation of the relief of the skin area.

In one particular aspect of the invention, the data control processing unit (CPU) makes it possible to determine the characterization of the parameter representing the cellulite, which comprises the fat and water content percentage in a unit volume of the skin area. Indeed, one may consider that cellulite is due not only to the accumulation of fat beneath the surface of the skin, but also to water retention in the skin surface.

In one particular aspect of the invention, the electrodes protrude to the exterior of the casing of the apparatus. This protruding state is suggested at least while the apparatus is operating, and allows the electrodes to come effectively into contact with the skin, such that only said electrodes are in contact, without the apparatus casing being able to touch the skin. This is all the more effective if the operating apparatus is moved in contact along the skin surface. The electrodes protrude from the casing by at least 0.5 mm, or even at least 50 mm.

In one particular aspect of the invention, the electrode material is a metal selected from the group consisting of stainless steel, indium tin oxide (ITO), titanium nitride (TiN), gold and carbon. These are metals with good electrical conductivity, and in some cases even good optical transparency (ITO).

In one particular aspect of the invention, the electrodes of the probe each have a skin-contact surface are value of between 50 mm² and 20 cm². Alternatively, as discussed below, the electrodes may form points with a diameter of between 0.1 mm and 1.5 mm, for example between 0.5 mm and 1.5 mm, in the form of a punch.

In one particular method of the invention, the probe comprises two electrode pairs, each with a skin-contact surface that is square or rectangular in shape. These shapes have better injection values for the measurement of fat. This can be used in quadri-polar mode for a comprehensive acquisition of the area being examined. In this method, the electrode pairs are either, square or rectangular.

In one particular method of the invention, as an alternative to the previous one, the probe comprises a row or at least two crossed rows or a matrix of various electrodes. These electrodes may then be pointed in shape.

In the case of a matrix, this probe is referred to as a “biochip.” The electrodes are pointed in shape. These points are non-invasive and of a length on the order of a few millimeters. The points have a skin-contact surface that is disc-shaped, and the diameter of the disc is, in total, between 0.5 mm and 1.5 mm.

In one particular aspect of the invention, at least one electrode of the probe is detachable. This facilitates their cleaning and reuse. This can allow for interchanging them with new, identical or different electrodes, to adjust the desired measurement and to increase the lifespan of the apparatus.

In one particular aspect of the invention, the value of the spacing between the electrodes is within the range [6 mm; 30 mm]. The spacing between the electrodes, and particularly the injecting electrodes, is important for adjusting the depth of analysis of the skin area. The injecting electrodes are preferably inside the receiving electrodes,

In one particular aspect of the invention, the electrodes are assembled to be floating and/or the electrode support is flexible. In both cases, this makes it possible to optimize contact with the surface of the skin area, particularly when it has pronounced curves. This makes it possible to control the contact pressure, which is particularly appreciated when the apparatus can be used in a portable manner along the skin surface while running the apparatus over it. In one particular aspect of the invention, the apparatus may include a means of adjusting the spacing between the electrodes of the probe. The spacing is adjustable between at least two injecting electrodes.

In one particular aspect of the invention, at least one electrode can be retracted into the casing, so as to assume at least one measurement position outside of the casing and one rest position inside the casing. The electrode(s) may be retracted manually or automatically to allow for the optimal storage of the apparatus, and for protecting the electrodes while storing or moving the apparatus when not in operation. This may make it possible to use only a portion of the electrodes and also to adjust the spacing between the protruding “operational” electrodes. The electrodes may be retractable jointly by pair, and the retractability may be permitted before or after, or even during the measurement session. If this apparatus is integrated in a skin treatment device, as will be discussed below, this will allow the electrodes to be operational only for a portion of the treatment session, for example just at the beginning of the treatment or before it begins.

In one alternative method of implementation allowing for several measurements by a probe with multiple electrodes, several pairs of electrodes are provided, arranged for example along a same line (1D) or arranged as a matrix (2D) and comprising a switch that makes it possible to form different configurations of injecting electrode pairs and/or receiving electrode pairs, however without requiring certain electrodes to be retractable. In one particular aspect of the invention, the probe can be retracted into the casing. This procures the same advantages as previously mentioned and easy use in only two positions of the probe.

In one particular aspect of the invention, the apparatus comprises a means of detecting the pressure of the skin surface and a means of allowing the measurement to be taken when the pressure detected exceeds a predetermined threshold. This makes the measurements safer and more effective. The electrical system is repeatable and satisfactory (bi-polar loop). This may be used in both static and dynamic usage.

In one particular aspect of the invention, the apparatus comprises a means of detecting the speed of movement over the skin surface, and a means of allowing the measurement to be taken when the detected speed is within a predetermined range. This is used if the measurement apparatus is able to measure while in motion along the skin surface. It may be necessary to keep the speed as constant as possible, or if it is possible to ascertain the speed value, it will be used for control in a massage device comprising the measurement apparatus.

In a second facet of the invention, we will describe a skin treatment device that includes, in a built-in manner, the mobile autonomous measurement and analysis apparatus.

This skin treatment device has a casing comprising skin treatment components, a control processing unit (CPU) for said treatment components, the skin surface measurement and analysis apparatus described in the invention, said control processing unit (CPU) being able to automatically control the skin treatment components based on at least one parameter representing the fat measured and/or calculated by the measurement and analysis apparatus. Being built-in in this way may make it possible, based on measurements and/or calculations from the session in progress, and even also other sessions, to adjust the treatment apparatus prior to its startup in skin treatment mode, and may make it possible to establish a servo-control loop during the treatment based on measurements and/or calculations from the session in progress, and even also other sessions.

In one particular aspect of the invention, the skin treatment device comprises, as components for the treatment, two rollers with parallel respective longitudinal axes and/or a suction chamber, and has at least one of said electrode pairs adjacent to at least one portion of the treatment components. The phrase “electrode adjacent to least one portion of the treatment components” means the existence of a smaller distance between the electrode and the smallest possible part of the treatment components, while taking into account production limitations. This distance may be on the order of a few millimeters.

In one particular aspect of the invention, the skin treatment device comprises an interface and a processing and control unit that saves the measurements and the calculation results for each session, and displays on the interface, from the analysis of these measurements and results, at least one suggested parameter setting for the operation of the apparatus from among the following: suction level, activation of the motorized spinning of the roller(s), the spin direction and/or speed of the roller(s), the duration of the session, changes in the operating parameters of the device from one session to another and the spacing between sessions.

Moreover, various other characteristics of the invention are apparent in the detailed description below, in reference to the attached drawings, which illustrate in a non-limiting manner one method of implementing an apparatus described in the invention.

FIG. 1 presents a diagram of an existing principle of bioelectric impedance measurement based on the use of four electrodes;

FIG. 2 presents a diagram of an existing four-electrode measurement device;

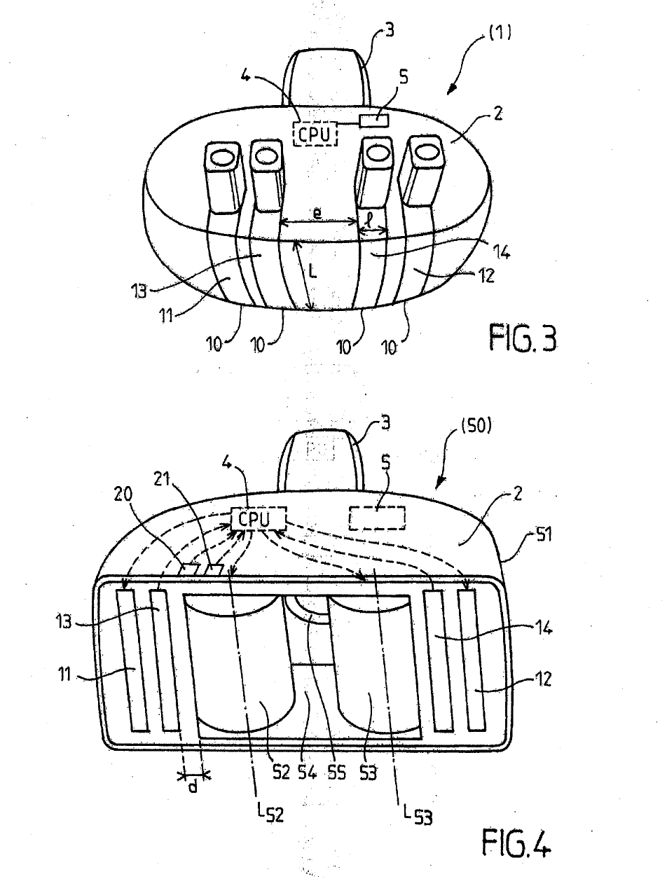

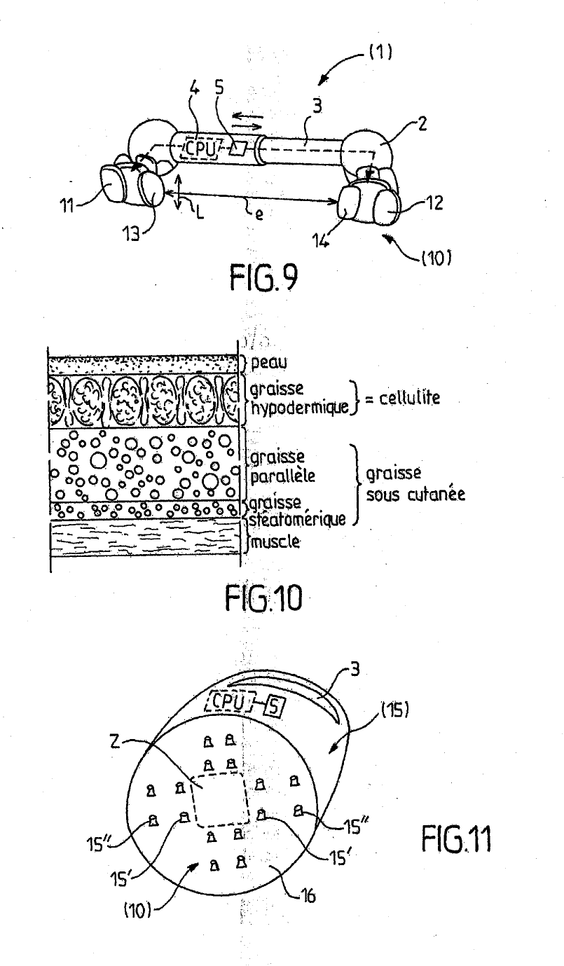

FIGS. 3 and 9, respectively, are perspective drawings of a first method of implementing a measurement apparatus described in the invention, as a first alternative, and a second grip-type alternative, respectively; FIG. 7 is a perspective drawing of a second method of implementing a measurement apparatus described in the invention;

FIG. 4 is a perspective drawing of a skin treatment apparatus including a measurement apparatus described in the first method of the invention;

FIG. 5 is a perspective drawing of a skin treatment apparatus including a measurement apparatus described in the invention;

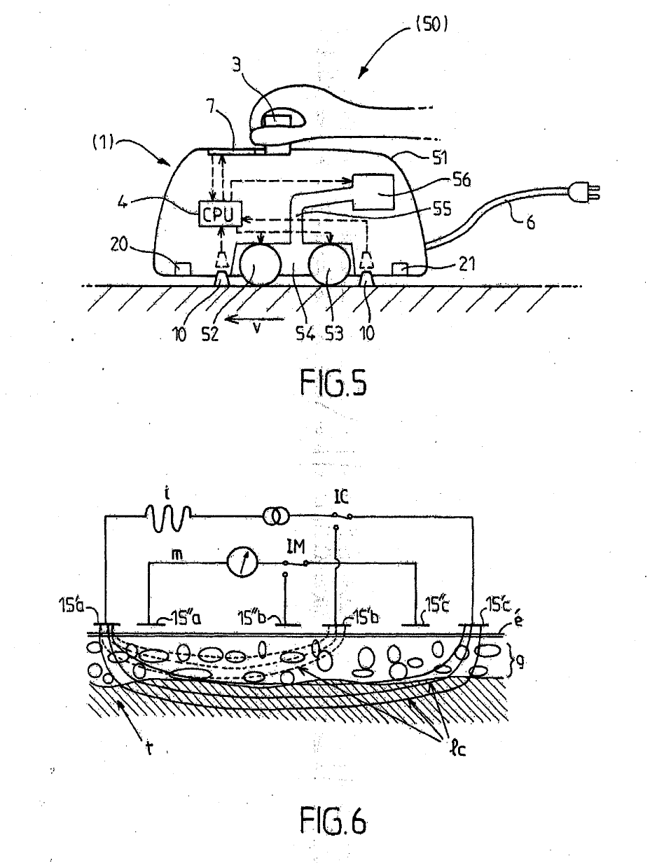

FIG. 6 is a cross-section illustration of one particular method of a probe with three groups of electrodes;

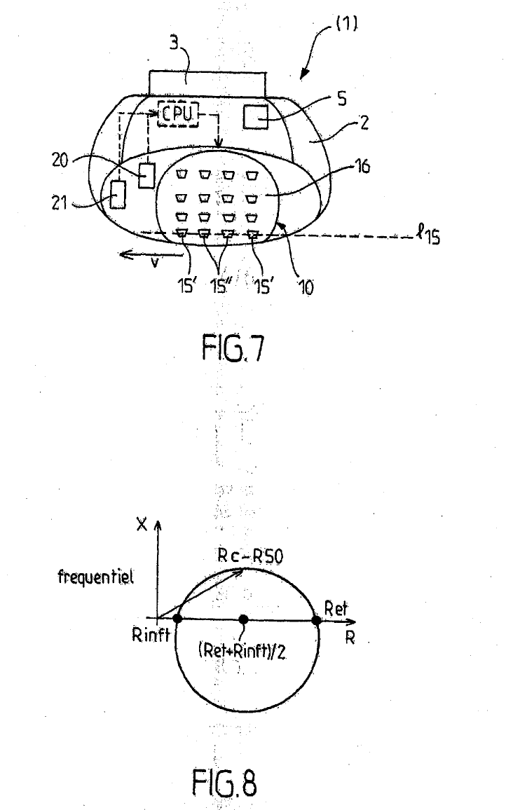

FIG. 8 is a bio-impedance graph with the resistance plotted on the x-axis and the reactance on the y-axis;

FIG. 10 shows a cross-section of tissue;

FIG. 11 is a perspective view of a third method of implementing a measurement apparatus described in the invention;

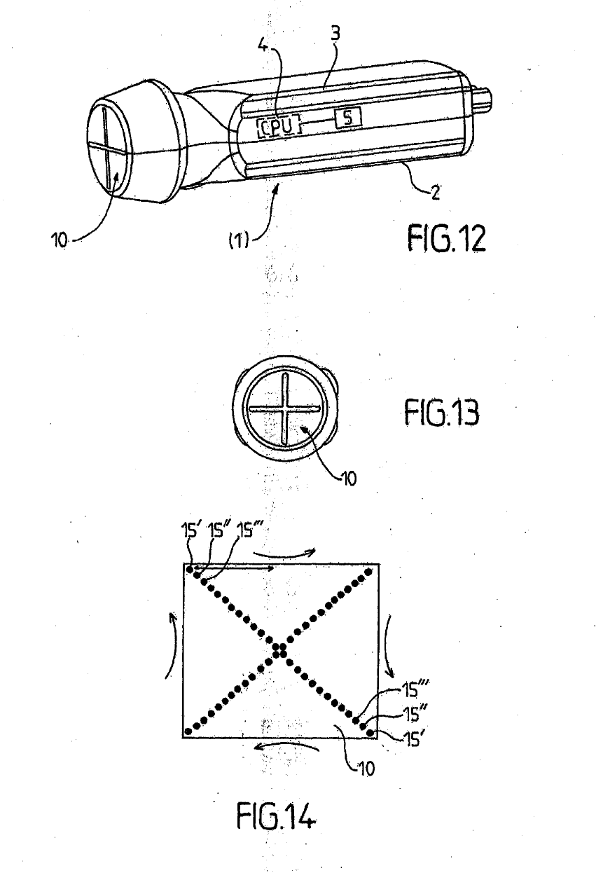

FIGS. 12 and 13 present a perspective view and a front view of a fourth method of implementing a measurement apparatus described in the invention, and FIG. 14 depicts the measurement electrodes arranged in an X formation.

### DETAILED DESCRIPTION

Here we will describe the invention in greater detail. For both methods of implementing the measurement apparatus depicted in FIGS. 3 and 7, the reference numbers used are the same for equivalent components. The reference numbers are repeated when the measurement apparatus is built directly into a single casing comprising a skin treatment apparatus, as illustrated in FIG. 4 or 5.

FIGS. 3, 7, 9, 11, 12, 13 and 14 depict the autonomous mobile apparatus (1) for measuring and analyzing at least one parameter representing cellulite of a skin surface area comprising at least one probe (10). The probe comprises at least two pairs of electrodes intended to come into contact with the skin surface area defining a first pair of “injecting” electrodes (11, 12), and a second pair of “receiving” electrodes (13, 14) for measuring the response to the injection of the variable or alternating current. The autonomous apparatus is designed to be applied to the skin and comprises, for good ergonomics, a handle-type gripping means (3) on top of the casing (2 or 51). Alternatively, the apparatus may, itself, take on the form of a handle or a grip as illustrated in FIG. 9. This grip or handle may be telescoping in the place where it is gripped, in a roughly tubular shape, to allow for adjusting the overall distance between each electrode pair. This grip or handle may be flexible, and the handle head(s) may be assembled on a ball and socket: the curve of the handle or grip depends on and adapts to the shape of the person (thin, muscular or obese). The handle or grip is used to measure the cellulite, and/or the parallel fat and/or the steatomeric fat. The apparatus may operate statically on the skin or dynamically by moving it along the skin.

The power source for the apparatus may be built-in, for example a photovoltaic cell or battery, or the device may be plugged into the power grid by a sector-6 power cord illustrated in FIG. 5. It makes it possible to produce, in the first injecting electrode pair (11, 12), an electrical signal of variable intensity (i) within the range [60 μA; 4000 μA] and of a frequency within the range [1 Hz; 1000 kHz], and in one particular example, between [1 kHz; 1000 kHz]. A data control processing unit (CPU) makes it possible to determine the information pertaining to bio-impedance (Z) of said area, and said at least one parameter representing the cellulite of the skin surface area. The control processing unit (CPU) indicated by reference number 4 covers a unit for calculating and processing data/nom the measurements taken, a unit for saving the measurements and results and any other data pertaining to the skin treatment apparatus with the built-in measurement apparatus described in the invention, for example data pertaining to the operation suggested by the interface.

The characterization of cellulite is not currently internationally standardized. The inventors have therefore had to create a system of characterizing this result that can be visible and understood by the user and that can show a progression. The characterization is calculated based in particular on bio-impedance measurements in the skin area in question.

In a first example, the characterization of the cellulite parameter comprises a natural integer on a scale between 0 and n, representing the stage of progression of the celluiite. The scale in this case is defined as being from 0 to 10 for example, but could be a smaller range, such as 0 to 5, for example, or larger. The term “dimpling score” can be used, which the user will be able to understand immediately and the development of which can be monitored over time. Of course, the characterization of no cellulite is 0 in this case.

In the second and third examples, in a different manner, it is a matter of providing a representation of the relief of two different areas. In a second example, the characterization of the cellulite parameter comprises the length, and even the overall size, of the hypodermal attachment for a unit length, and even a unit surface area of the skin area. The characterization of no cellulite in this case is 1, as the attachment is flatter if less cellulite is measured. This is a cross-section of internal relief, in a third example, the characterization of the cellulite parameter is the surface roughness of the skin area. This is external relief.

In a fourth example, the characterization of the cellulite parameter comprises the fat and water content percentage in a unit volume of the skin area.

As shown in FIGS. 3, 4 and 5, the electrodes (11, 12, 13, 14 and 15) protrude to the exterior of the casing (2 or 51) of the apparatus (1 or 50). Reference number 15 also pertains to reference numbers 15′, 15″, 15′a, 15′b, 15′c and 15″c, explained according to different methods of implementing the probe, as illustrated in FIGS. 7 and 11.

The material of the electrodes (11, 12, 13, 14 and 15) is a metal selected from among the group consisting of stainless steel, indium tin oxide, titanium nitride, gold and carbon, either alone or in combination. For the second method of the invention, the probe (10) illustrated in FIG. 7 is referred to as a “biochip” and the electrodes are made of titanium nitride.

In a first method of implementing the probe comprising two pairs of electrodes (11, 12, 13, 14 and 15), they each have a skin-contact surface area value of between 50 mm² and 20 cm². Ideally, their skin-contact surface area is square or rectangular in shape. In greater detail, the length (L) of the four rectangles, as illustrated, may be between 10 and 50 mm, and their width between 5 and 40 mm. There may be many pairs of this type for taking a measurement over a larger skin surface.

In the second method of implementing the probe (10), it includes a matrix of multiple pointed electrodes. This type of probe is, for example, referred to as a “biochip” in tri-polar mode for ascertaining the characteristics at a specific point, and thus mapping the area in question. The probe may be a regular matrix, as illustrated in FIG. 7, where the electrodes (15) operate in pairs: the injecting electrodes (15′) facing one another operate with the pair of receiving electrodes facing one another in the same alignment. The measurements are over a same line (115) but may be in an X formation in this method. Each electrode pair is then used differently depending on the desired measurement, and the results may be advantageously combined. As an example of spacing, there are 8 mm between the receiving electrodes to reach a depth of 4 mm beneath the skin; it was observed by the inventors through multiples tests that this depth beneath the skin is located generally beyond the dermis in the area where cellulite starts. Single-frequency measurement duration: One may begin at 150 ms and in multiple frequencies complete with an ascertaining algorithm one can go up to 30 seconds, in theory. The biochip electrodes may be controlled individually. Whatever electrodes are used, the measurement is taken at a depth of about 1 cm, from 0.1 mm to 30 mm for the biochip, and from 2 to 30 mm, and in particular between 6 mm and 30 mm for traditional electrodes. There may be from 4 to 64 electrodes or more when a biochip is used. The electrodes form, for example, a 9×8 matrix comprising 64 recording electrodes, 4 reference electrodes and 4 mass electrodes, the spacing between the electrodes being from 400 microns to 700 microns, and the electrode diameter around 100 microns. The base material may be a sheet of flexible polyimide. There exists, for example, a biochip marketed by the company Multi Channel Systems under product reference number FlexMEA72.

Alternatively, the probe may comprise more than four electrodes, and as illustrated in FIG. 6, six electrodes. When a potential is established between two electrodes (15′a and 15′b), the field lines (Ic) will be distributed along the areas with the least electrical resistance. As described above, the source may have a square or sinusoidal signal.

Fatty tissue is very resistant compared to muscle tissue. The tat layer comprises several kinds of fat, as illustrated in FIG. 10: starting from the skin, the first layer is the hypodermal fat or cellulite, then the second layer is the parallel fat, and the third layer is the steatomeric fat, the second and third layers forming the subcutaneous fat layer. When the fat layer is significant and the electrodes (15′a and 15″b) are far apart, the electrical flow will have a tendency to pass through the muscle layer, and thus the tissue through which it passes is not representative of the superficial layer. However, if the electrodes are too close to one another, a superficial current may be established that is not representative of the deep layer. It is particularly difficult, in order to measure the cellulite layer only or the subcutaneous fat layer only, to determine the intensities, combined with the frequencies and spacing between the electrodes, in the most reliable possible manner for a significant number of people.

Thus, the manner of arranging electrodes on the surface of the skin for evaluating the distribution of the electrical field lines and finding the volume of living tissue in question through measurement must be judiciously established. Several electrode pairs (15′a and 15″a; 15″b and 15′b; 15″c and 15′c, etc.) are arranged over the area of skin being measured that is in contact with the apparatus. The measurement principle consists of making multiple simultaneous electrode switches (switches labeled “IC” and “IM” with C for Current and M for Measurement) while keeping the electrode reference numbers 15′a and 15″a. By increasing the number of electrode pairs on the skin surface, it is possible to identity the distance between electrodes for which the electrical field lines will encounter the muscle area, which is more electrically-conductive than the fat layer. Starting from that distance, resistance increases less quickly as a function of the distance between the electrode pairs. Given the distance between the electrodes characteristic of a change in electrical conduction, it is then possible to determine the volume of the fat layer of the dermis being measured, as an increased distance between electrode pairs is indicative of a thicker layer.

At least one electrode (11, 12, 13, 14 and 15) of the probe (10) may be detachable, and even interchangeable. Alternatively, a portion of the probe, and specifically one or more electrodes may also be.

in FIG. 3, the spacing value (e) between the receiving electrodes (13, 14) and those of the ensemble (15) is within the range [0.1 mm; 30 mm], and even within the sub-range [6 mm; 30 mm], or within the range [6 mm; 15 mm], as it relates directly to the measurement depth in the volume of skin being measured.

FIG. 11 depicts a perspective view of a third method of implementing a measurement apparatus described in the invention. It still involves electrode pairs (15′, 15″), which this time are crossed, or in an X formation, in order to measure the cellulite of a given skin area (z).

Finally, FIGS. 12, 13 and 14 all depict a fourth method of implementing a measurement apparatus described in the invention, which we will refer to as a “punch.” The punch is in an X formation, formed by two rows of several electrodes (15′, 15″, 15′″, etc.) arranged perpendicularly and crossing in the middle. One can imagine several crossed rows. In the example depicted, there are 13 electrodes in half-rows, but the number may vary, of course. The punch in this example may explore the fat layer located between 3 to 15 mm beneath the skin. The receiving electrodes must be separated at 2*e, e being the length separating the receiving electrodes, thus the distance is between 6 and 30 mm, One may wish to have an exploration step at a depth of 1 mm, which is a spacing step between the receiving electrodes of 2 mm. The measurement series resembles that of the method depicted in FIG. 6, hut the measurement is taken over two rows to produce a partially three dimensional perspective.

Thus, there exist several methods of calculation, including the measurement known as Focus Impedance Measurement (FIM). This method makes it possible to overcome sensitivity phenomena related to the proximity of test electrode-points, which are nevertheless very close to one another, and to thus obtain the most reliable measurement. Moreover, by playing with the spacing between the points, one can analyze the dermis in thin layers. The FIM method is already known and has been the subject of two reference publications: A new technique with improved zone localization, K. S. Rabbani, M. Sarker, M. H R. Akond . . . —Annals of the New York . . . , 1999—Wiley Online Library, A new four-electrode Focused Impedance Measurement (FIM) system for physiological study, K. S. Rabbani, M. A. S. Karal—Annals of biomedical engineering, 2008—Springer. For a complete description, please refer to these two publications.

In general, measurements are obtained with dry electrodes, but the use of a gel may be considered.

Moreover, it has been noted that a tri-polar probe is advantageous for obtaining measurement information at a precise point. Repeatability is very good.

The electrodes are assembled to be floating and/or the electrode (11, 12, 13, 14 and 15) support is flexible. For example, the electrodes may be assembled on a spring. The spring may be, for example, in the tip. The stroke in question may be several dozen or several hundred microns, for example approximately 500 microns. For example, in an alternative or additional manner, the electrode support may be flexible or soft, such as silicone or soft textile, for example. A conductive textile material may be used, and in particular a sheet of polyimide or an elastic material, such as silicone, loaded with or containing a metal contact.

The apparatus may comprise a means of adjusting the spacing between the electrodes (11, 12, 13, 14 and 15) of the probe. The spacing can be adjusted between at least two injecting electrodes, not between two receiving electrodes. One may provide for an assembly of electrodes on mobile crosspieces that can be moved closer together or farther apart simultaneously.

As depicted in FIG. 6, at least one electrode of the probe may be retractable into the casing (2) so as to assume at least one measurement position outside of the casing (solid lines) and one rest position inside the casing (dotted lines). The entire probe (10) may be retractable into the casing. A means of detecting the pressure (20) of the skin surface is provided, as well as a control processing unit (4) for allowing the measurement to be taken when the pressure detected exceeds a predetermined threshold previously saved in the control processing unit. The pressure detection means may include one pressure detector or two separate pressure detectors alongside the electrodes to verify contact during a sufficient interval of time to take the measurement. In addition, a means of detecting the speed (21) of movement over the skin surface and a means of allowing the measurement to be taken when the speed detected is within a predetermined range. As illustrated in FIGS. 4 and 5, the invention also pertains to a skin treatment device (50) with a casing (51) comprising skin treatment components (52, 53, 54, 55 and 56), a control processing unit (4) for said treatment components (52, 53, 54, 55 and 56), the apparatus (1) for measuring and analyzing the skin surface as described in the invention, said control processing unit (4) being able to automatically control the skin treatment components (52, 53, 54, 55 and 56) based on at least one parameter measured and/or calculated by said autonomous measurement and analysis apparatus (1). In one example, the treatment components (52, 53, 54, 55, 56) are two rollers (52, 53) with respective parallel longitudinal axes (L52, L53) and/or a skin suction chamber (54), and at least one of the electrode pairs is as adjacent as possible (a few millimeters) to at least one part of the massage components (52, 53), yet without touching them. The suction chamber (54) is created by means of a depression source (56), which may be integrated in a part that can be gripped by the user or may be placed elsewhere in a base and connected via a suction channel (55). In the example illustrated, the suction chamber is between the rollers, but it may also be located outside of the two rollers. The rollers may spin freely, or also even be slowed, and/or be spun by one or more motors (not depicted).

The skin treatment device has an interface (7) and a control processing unit (4) that stores the measurements and calculation results for each session, and displays on the interface (7), based on an analysis of these measurements and results, at least one suggested parameter setting for the operation of the apparatus from among the following: suction level, activation of the motorized spin of the roller(s), the spin direction and/or speed of the roller(s), the duration of the session, changes in operational parameters from one session to another and the spacing of the sessions,

The interface consists of a display and/or LEDs for communicating the input data, measured data, bio-impedance, cellulite, apparatus setting suggestions and proposed recommendations; hut it may also comprise buttons or a touch screen for selecting the proposal or manually modifying the proposals calculated by the device.

Of course, other modifications may be made to the invention within the scope of the attached claims.

In reference to FIG. 6:

The symbol i=current source signal
The symbol IC=Switch C
The symbol IM=Switch M
The symbol m=measurement
The symbol t=electrically conductive tissue
The symbol é=epidermis
The symbol Ic=electric field lines

1. Autonomous mobile apparatus (1) for measuring and analyzing at least one parameter representing the fat of a skin surface area, comprising:
At least one probe (10) comprising at least two electrode pairs intended to come into contact with the skin surface area defining a first pair of injecting electrodes (11, 12) and a second pair of receiving electrodes (13, 14) for measuring the response to the injection;
A power source (5) for producing in the first pair of injecting electrodes (11, 12), a variable electrical signal with a variable current intensity (i) within the range of 60 μA to 4000 μA and with a frequency within the range of 1 kHz to 1000 kHz;
A data control processing unit (CPU) for determining the information related to the bio-impedance (Z) of said area, with said at least one parameter representing the fat in the skin surface area.

2. Apparatus described in claim 1, in which said variable electrical signal with current intensity (i) is a sinusoidal alternating signal as a function of time.

3. Apparatus described in claim 1, in which said variable electrical signal with current intensity (i) is a rectangular signal, such as a square signal, as a function of time.

4. Apparatus described in claim 1, in which the parameter is the subcutaneous fat and the data control processing unit (CPU) makes it possible to determine the quantity of subcutaneous fat that is equal or proportional to:
A*((Ret+Rinft)/2)−B
In which Ret is the resistance extrapolated to a null frequency,
Rinft is the resistance extrapolated to an infinite frequency,
And A and B are constants.

5. Apparatus described in claim 1, in which the parameter is cellulite and the data control processing unit (CPU) makes it possible to determine the thickness of the cellulite being equal or proportional to:
Ln (Rinft/C)/D
In which Rinft is the resistance extrapolated to an infinite frequency, and in which C and D are constants.

6. Apparatus described in claim 1, in which the data control processing unit (CPU) makes it possible to determine a characterization of the parameter representing cellulite and that comprises a natural integer on a scale between 0 and n, representing a stage of progression of the cellulite.

7. Apparatus described in claim 1, in which the data control processing unit (CPU) makes it possible to determine a characterization of the parameter representing cellulite, comprising a length of a hypodermal attachment for a unit length of the skin area, or the hypodermal attachment size for a unit surface area of the skin area.

8. Apparatus described in claim 1, in which the data control processing unit (CPU) makes it possible to determine a characterization of the parameter representing cellulite and being a surface roughness of the skin area.

9. Apparatus described in claim 1, in which the data control processing unit (CPU) makes it possible to determine a characterization of the parameter representing cellulite, which comprises a fat and water content percentage in a unit volume of the skin area.

10. Apparatus described in claim 1, in which the electrodes (11, 12, 13, 14 and 15) protrude to an exterior of an apparatus casing.

11. Apparatus described in claim 1, in which the material of the electrodes (11, 12, 13, 14 and 15) is a metal selected from among the group consisting of stainless steel, indium tin oxide, titanium nitride, gold and carbon.

12. Apparatus described in claim 1, in which the electrodes (11, 12, 13, 14 and 15) of the probe each have a skin-contact surface area value of between 50 mm2 and 20 cm2.

13. Apparatus described in claim 1, in which the probe (10) comprises two electrode pairs (11, 12, 13, 14 and 15) each with a skin-contact surface area that is square or rectangular in shape.

14. Apparatus described in claim 1, in which the probe (10) comprises one row or at least two crossed rows or a matrix of multiple electrodes.

15. Apparatus described in claim 1, in which the electrodes are pointed shape.

16. Apparatus described in claim 1, in which each electrode has a skin-contact surface in the form of a disc, a diameter of which is between 0.5 mm and 1.5 mm.

17. Apparatus described in claim 1, in which at least one electrode (11, 12, 13, 14 and 15) of the probe (10) is detachable.

18. Apparatus described in claim 1, in which a spacing between the receiving electrodes (11, 12, 13, 14 and 15) is within the range of 6 mm to 30 mm.

19. Apparatus described in claim 1, in which the electrodes are assembled to be floating and/or the electrode (11, 12, 13, 14, and 15) support is flexible.

20. Apparatus described in claim 1, comprising a means of adjusting the spacing between electrodes (11, 12, 13, 14 and 15) of the probe.

21. Apparatus described in claim 1, wherein at least one electrode (11, 12, 13, 14 and 15) can be retracted into the casing (2) so as to assume a measurement position outside of the casing and a rest position inside of the casing.

22. Apparatus described in claim 1, wherein the probe (10) can be retracted into the casing.

23. Apparatus described in claim 1, comprising a means of detecting a pressure (20) of the skin surface and a means (4) of allowing a measurement to be taken when the pressure detected exceeds a predetermined threshold.

24. Apparatus described in claim 1, comprising a means of detecting a speed (21) of movement over the skin surface and a means of allowing a measurement to be taken when the speed detected falls within a predetermined range.

25. Skin treatment device (50) with a casing (51) comprising:
Skin treatment components (52, 53, 54, 55 and 56),
A control processing unit (4) for said treatment components (52, 53, 54, 55 and 56),
The apparatus (1) for measuring and analyzing the skin surface as described in claim 1,
Said control processing unit (4) being able to automatically control the skin treatment components (52, 53, 54, 55 and 56) based on at least one parameter representing the fat measured and/or calculated by said measurement and analysis apparatus (1).

26. Skin treatment device (50) as described in claim 25, comprising, as treatment components (52, 53, 54, 55 and 56), two rollers (52, 53) with respective parallel longitudinal axes (L52, L53) and/or a suction chamber (54), wherein at least one of said electrode pairs (11, 13; 12, 14 and 15) is adjacent to at least one part of the treatment components (52, 53).

27. Skin treatment device described in claim 26, comprising an interface (7) and a control processing unit (4) that saves measurements and calculation results for each treatment session and displays on the interface (7), based on the analysis of these measurements and results, at least one proposed operational parameter setting for the apparatus from among the following:
Suction level;
Activation of the motorized spinning of the roller(s);
Spin direction and/or speed of the roller(s);
Duration of the session;
Changes in the operating parameters of the device from one session to another;
Spacing between sessions.
